(19) United States
(12) Patent Application Publication
(10) Pub. No.: US 2018/0360727 A1
(43) Pub. Date: Dec. 20, 2018

(54) COSMETIC PROCESS FOR TREATING KERATIN MATERIALS WITH AN ACRYLIC POLYMER BEARING MALEIC ANHYDRIDE GROUPS

(71) Applicant: L'OREAL, Paris (FR)

(72) Inventors: Bertrand LION, Aulnay-sous-Bois (FR); Julien PORTAL, Aulnay-sous-Bois (FR)

(21) Appl. No.: 16/062,878

(22) PCT Filed: Dec. 16, 2016
(86) PCT No.: PCT/EP2016/081347

§ 371 Date: Jun. 15, 2018

(30) Foreign Application Priority Data
Dec. 22, 2015 (FR) 1563111

(51) Int. Cl. A61K 8/81; A61K 8/898; A61K 8/41; A61K 8/31; A61Q 1/06; A61Q 1/10; A61Q 3/00; A61Q 5/06; A61Q 19/00

(52) U.S. Cl. CPC A61K 8/8141; A61K 8/898; A61K 8/41; A61K 8/31; A61Q 1/06; A61K 2800/95; A61Q 3/00; A61Q 5/06; A61Q 19/00; A61K 2800/43; A61K 2800/884; A61Q 1/10

(57) ABSTRACT

The invention relates to a cosmetic process for caring for or making up keratin materials, comprising the sequential application to the keratin materials: of a cosmetic composition comprising a maleic anhydride acrylic polymer obtained by polymerization of: (a) 50% to 90% by weight, relative to the total weight of monomers, of isobornyl (meth)acrylate (b) 1% to 50% by weight of maleic anhydride (c) 0% to 49% by weight of additional (meth)acrylate monomer; and of a polyamine compound bearing several primary amine and/or secondary amine groups. The process makes it possible to obtain a film-forming deposit that has good resistance to water, to oil and to sebum. The film is also non-tacky and transfer-resistant.

The present invention relates to a cosmetic process for treating keratin materials using a maleic anhydride acrylic polymer and a polyamine compound, and also to a kit for performing said process.

Cosmetic products often require the use of a film-forming polymer to obtain a deposit of the product on keratin materials that has good cosmetic properties. In particular, it is necessary for the film-forming deposit to have good persistence, in particular for the deposit not to transfer during contact with the fingers, clothing, a glass or a cup, and also good persistence on contact with water, especially rain or during showering or alternatively perspiration. Skin sebum may also damage the film-forming deposit.

It is known to those skilled in the art to use polymers in order to obtain these good persistence properties throughout the day. These polymers are of very different chemical nature and are generally conveyed either in a fatty phase or in an aqueous phase. Examples that may be mentioned include silicone resins, polyacrylates and latices.

Although these polymers do indeed afford persistence properties, in particular transfer resistance, they may have a certain level of discomfort: for example, after applying the product, they may have a tacky aspect.

There is thus still a need for polymers that can afford good persistence properties while at the same time maintaining a certain level of comfort during use.

The inventors have discovered that a particular maleic anhydride acrylic polymer combined with a particular polyamine compound makes it possible to obtain a deposit on keratin materials that has good film-forming properties.

The film-forming deposit obtained has good water resistance and also good resistance to oil (especially to olive oil) and to sebum.

This particular acrylic polymer is readily conveyable in a hydrocarbon-based oil such as isododecane.

Furthermore, the film-forming deposit has good tack-resistance and transfer-resistance properties, especially when the film is touched with the fingers: the deposit obtained thus has good persistence properties.

Furthermore, when the maleic anhydride acrylic polymer is formulated with a non-volatile oil (often used in makeup products), for instance 2-octylethanol, the process according to the invention makes it possible to obtain a film-forming deposit which has good persistence, transfer-resistance, tack-resistance, water-resistance, oil-resistance and sebum-resistance properties.

This maleic anhydride acrylic polymer combined with said polyamine compound forms a film-forming deposit that is suitable for making up the skin or the lips or the eyelashes, such as foundations, lipsticks or mascaras, or for fixing the hair.

More precisely, a subject of the present invention is a treatment process, especially a cosmetic treatment process, in particular for caring for or making up keratin materials, comprising the sequential application to keratin materials of a composition, especially a cosmetic composition, comprising a maleic anhydride acrylic polymer and a polyamine compound, or a cosmetic composition containing same,

said maleic anhydride acrylic polymer being able to be obtained by polymerization of:

(a) 50% to 90% by weight, relative to the total weight of monomers, of isobornyl (meth)acrylate

(b) 1% to 50% by weight of maleic anhydride

(c) 0 to 49% by weight of additional (meth)acrylate monomer chosen from:

(i) linear or branched, saturated or unsaturated C₁-C₂₀ alkyl (meth)acrylates, optionally interrupted with one or more non-adjacent heteroatoms chosen from O and S or with a group NR, R being a C₁-C₄ alkyl group, optionally substituted with a phenyl or furfuryl group;

(ii) saturated C₄-C₈ cycloalkyl (meth)acrylates optionally interrupted with O or NH;

said amine compound being chosen from polyamine compounds bearing several primary amine and/or secondary amine groups, the amine compound not being an alkoxysilane.

The process according to the invention is suitable for caring for or making up keratin materials, such as the skin, the lips, the eyelashes, the hair or the nails.

The process according to the invention is also suitable for shaping the hair, especially for styling. The hair fixing shows good water resistance.

A subject of the invention is also a kit comprising a first composition comprising said maleic anhydride acrylic polymer as described previously and comprising a physiologically acceptable medium, and a second composition comprising an amine compound as described previously and comprising a physiologically acceptable medium, the first and second compositions each being packaged in a separate packaging assembly.

The composition packaging assembly is, in a known manner, any packaging that is suitable for storing cosmetic compositions (in particular a bottle, tube, spray bottle or aerosol bottle).

Such a kit allows the skin treatment process according to the invention to be performed.

The maleic anhydride acrylic polymer used according to the invention comprises (or is derived from the polymerization of) an isobornyl (meth)acrylate, maleic anhydride and optionally an additional acrylate monomer as defined previously. Advantageously, the maleic anhydride acrylic polymer is formed essentially from these monomers in the contents described hereinabove or hereinbelow.

Advantageously, the polymer used according to the invention is derived from the polymerization of:

(a) 50% to 90% by weight, relative to the total weight of monomers, of isobornyl (meth)acrylate

(b) 5% to 50% by weight of maleic anhydride

(c) 0 to 30% by weight of additional (meth)acrylate monomer as described previously.

Advantageously, the polymer used according to the invention is derived from the polymerization of:

(a) 50% to 80% by weight, relative to the total weight of monomers, of isobornyl (meth)acrylate

(b) 5% to 30% by weight of maleic anhydride

(c) 15 to 30% by weight of additional (meth)acrylate monomer as described previously.

Advantageously, the polymer used according to the invention is derived from the polymerization of:

(a) 50% to 80% by weight, relative to the total weight of monomers, of isobornyl (meth)acrylate

(b) 5% to 25% by weight of maleic anhydride

(c) 15 to 30% by weight of additional (meth)acrylate monomer as described previously.

Advantageously, the polymer used according to the invention is derived from the polymerization of:

(a) 50% to 80% by weight, relative to the total weight of monomers, of isobornyl (meth)acrylate

(b) 5% to 15% by weight of maleic anhydride

(c) 15 to 30% by weight of additional (meth)acrylate monomer as described previously.

Advantageously, the polymer used according to the invention is derived from the polymerization of:

(a) 60% to 80% by weight, relative to the total weight of monomers, of isobornyl (meth)acrylate

(b) 5% to 12% by weight of maleic anhydride

(c) 15 to 30% by weight of additional (meth)acrylate monomer as described previously.

Advantageously, the polymer used according to the invention is derived from the polymerization of:

(a) 60% to 80% by weight, relative to the total weight of monomers, of isobornyl (meth)acrylate

(b) 5% to 12% by weight of maleic anhydride

(c) 18 to 30% by weight of additional (meth)acrylate monomer as described previously.

The additional (meth)acrylate monomer is preferably chosen from C₆-C₁₆ alkyl (meth)acrylates, and preferentially chosen from C₆-C₁₆ alkyl acrylates.

As examples of C₆-C₁₆ alkyl (meth)acrylates, mention may be made of hexyl (meth)acrylate, octyl (meth)acrylate, 2-ethylhexyl (meth)acrylate, decyl (meth)acrylate and lauryl (meth)acrylate. 2-Ethylhexyl acrylate is preferably used.

Preferably, the polymer used according to the invention comprises, or consists of, isobornyl acrylate, 2-ethylhexyl acrylate and maleic anhydride.

A particularly preferred polymer used according to the invention is derived from the polymerization of:

(a) 50% to 80% by weight, relative to the total weight of monomers, of isobornyl (meth)acrylate

(b) 5% to 30% by weight of maleic anhydride

(c) 15% to 30% by weight of C₆-0₁₆ alkyl acrylate monomer.

A particularly preferred polymer used according to the invention is derived from the polymerization of:

(a) 50% to 80% by weight, relative to the total weight of monomers, of isobornyl (meth)acrylate

(b) 5% to 25% by weight of maleic anhydride

(c) 15% to 30% by weight of C₆-C₁₆ alkyl acrylate monomer.

A particularly preferred polymer used according to the invention is derived from the polymerization of:

(a) 50% to 80% by weight, relative to the total weight of monomers, of isobornyl (meth)acrylate

(b) 5% to 15% by weight of maleic anhydride

(c) 15% to 30% by weight of C₆-C₁₆ alkyl acrylate monomer.

A particularly preferred polymer used according to the invention is derived from the polymerization of:

(a) 60% to 80% by weight, relative to the total weight of monomers, of isobornyl (meth)acrylate

(b) 5% to 12% by weight of maleic anhydride

(c) 15% to 30% by weight of C₆-C₁₆ alkyl acrylate monomer.

A particularly preferred polymer used according to the invention is derived from the polymerization of:

(a) 60% to 80% by weight, relative to the total weight of monomers, of isobornyl (meth)acrylate

(b) 5% to 12% by weight of maleic anhydride

(c) 18% to 30% by weight of C₆-C₁₆ alkyl acrylate monomer.

Advantageously, the polymer used according to the invention consists of the monomers described previously.

Advantageously, the polymer used according to the invention is nonionic.

Preferably, the maleic anhydride acrylic polymer used according to the invention has a weight-average molecular weight ranging from 5000 to 1 000 000 g/mol, preferably ranging from 10 000 to 500 000 g/mol and preferentially ranging from 15 000 to 350 000 g/mol.

The molecular weight may especially be determined by steric exclusion chromatography, with THF eluent, polystyrene standard, 2414 refractometric detector from Waters.

The copolymer may be a random, alternating (block) or gradient polymer. Preferably, the copolymer is random.

The copolymer used according to the invention may be prepared by radical polymerization of the monomers described previously, especially as a mixture or added sequentially during the polymerization, especially using an organic solvent with a boiling point of greater than or equal to 60° C., for instance isododecane, ethanol, ethyl acetate, tetrahydrofuran, methyltetrahydrofuran or methyl ethyl ketone. The organic solvent makes it possible to dissolve the monomers used and the polymer formed.

The polymerization is especially performed in the presence of a radical initiator especially of peroxide type (for example tert-butyl peroxy-2-ethylhexanoate: Trigonox 21S; 2,5-dimethyl-2,5-bis(2-ethylhexanoylperoxy)hexane:Trigonox 141; tert-butyl peroxypivalate: Trigonox 25C75 from AkzoNobel) or of azo type, for example (AIBN: azobisisobutyronitrile; V50: 2,2′-azobis(2-amidinopropane) dihydrochloride).

The polymerization may be performed at a temperature ranging from 60 to 100° C., and preferably ranging from 60 to 85° C.

The polymerization time may be about 24 hours.

The polymer used according to the invention may be used in a composition comprising a physiologically acceptable medium, in particular in a cosmetic composition.

The term “physiologically acceptable medium” means a medium that is compatible with human keratin materials.

The term “cosmetic composition” is understood to mean a composition that is compatible with keratin materials, which has a pleasant colour, odour and feel and which does not cause unacceptable discomfort (stinging, tautness or redness) liable to discourage the consumer from using it.

The maleic anhydride acrylic polymer as defined previously may be present in the composition according to the invention in a content ranging from 0.1% to 40% by weight, relative to the total weight of the composition, preferably from 0.5% to 35% by weight of active material, preferentially ranging from 1% to 30% by weight, especially ranging from 5% to 30% by weight and more preferentially ranging from 10% to 30% by weight.

The polyamine compound used in the process according to the invention is chosen from polyamine compounds bearing several primary amine and/or secondary amine groups, the polyamine compound not being an alkoxysilane.

The term “alkoxysilane compound” means a compound comprising only one Si atom, this atom being bonded to at least one (especially from 1 to 3) group —OR, R being a linear or branched alkyl group, comprising from 1 to 6 carbon atoms.

According to a first embodiment of the invention, the polyamine compound is a compound comprising from 2 to 20 carbon atoms, in particular a non-polymeric compound. The term “non-polymeric compound” means a compound which is not directly obtained via a monomer polymerization reaction The polyamine compound is preferably a diamine compound.

Polyamine compounds that may be mentioned include N-methyl-1,3-diaminopropane, N-propyl-1,3-diaminopropane, N-isopropyl-1,3-diaminopropane, N-cyclohexyl-1,3-diaminopropane, 2-(3-aminopropylamino)ethanol, 3-(2-aminoethyl)aminopropylamine, bis(3-aminopropyl)amine, methylbis(3-aminopropyl)amine, N-(3-aminopropyl)-1,4-diaminobutane, N,N-dimethyldipropylenetriamine, 1,2-bis(3-aminopropylamino)ethane, N,N′-bis(3-aminopropyl)-1,3-propanediamine, ethylenediamine, 1,3-propylenedimaine, 1,4-butylenediamine, lysine, cystamine, xylenediamine, tris(2-aminoethyl)amine and spermidine. Preferably, the amine compound is chosen from ethylenediamine, 1,3-propylenediamine and 1,4-butylenediamine. Preferentially, the polyamine compound is ethylenediamine.

According to a second embodiment, the polyamine compound may be chosen from amine-based polymers.

The amine-based polymer may have a weight-average molecular weight ranging from 500 to 1 000 000, preferably ranging from 500 to 500 000, and preferentially ranging from 500 to 100 000.

As amine-based polymer, use may be made of poly((C₂-C₅)alkyleneimines), and in particular polyethyleneimines and polypropyleneimines, especially poly(ethyleneimine)s (for example the product sold under the reference 46,852-3 by the company Aldrich Chemical); poly(allylamine) (for example the product sold under the reference 47,913-6 by the company Aldrich Chemical); polyvinylamines and copolymers thereof, in particular with vinylamides; mention may in particular be made of vinylamine/vinylformamide copolymers such as those sold under the name Lupamin® 9030 by the company BASF; polyamino acids bearing NH₂ groups, such as polylysine, for example the product sold by the company JNC Corporation (formerly Chisso); aminodextran, such as the product sold by the company CarboMer Inc; amino polyvinyl alcohol, such as the product sold by the company CarboMer Inc, acrylamidopropylamine-based copolymers; chitosans; polydimethylsiloxanes comprising primary amine groups at the chain end or on side chains, for example aminopropyl side or end groups, for instance those of formula (A) or (B) or (C):

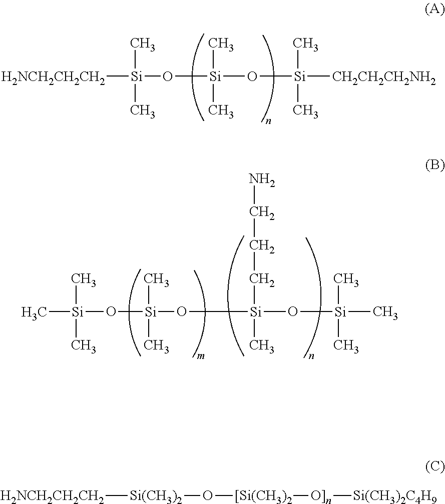

in formula (A): the value of n is such that the weight-average molecular weight of the silicone is between 500 and 55 000. As an example of aminosilicone (A), mention may be made of those sold under the names DMS-A11, DMS-Al2, DMS-A15, DMS-A21, DMS-A31, DMS-A32 and DMS-A35 by the company Gelest; reference 481688 from Aldrich.

in formula (B), the values of n and m are such that the weight-average molecular weight of the silicone is between 1000 and 55 000. As examples of silicone (B), mention may be made of those sold under the names AMS-132, AMS-152, AMS-162, AMS-163, AMS-191 and AMS-1203 by the company Gelest.

in formula (C), the value of n is such that the weight-average molecular weight of the silicone is between 500 and 3000. As an example of silicone (C), mention may be made of those sold under the names MCR-A11 and MCR-Al2 by the company Gelest;

amodimethicones of formula (D):

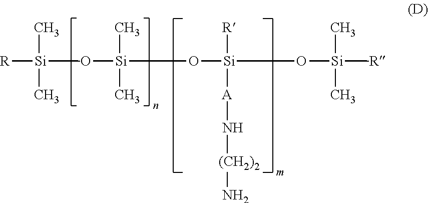

in which R, R′ and R″, which may be identical or different, each represent a C₁-C₄ alkyl or hydroxyl group, A represents a C₃ alkylene group and m and n are such that the weight-average molecular mass of the compound is between 5000 and 500 000 approximately;

amodimethicones of formula (K):

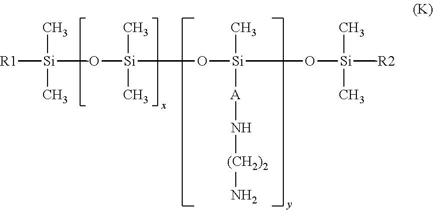

in which:
R1 and R2, which may be identical or different, preferably identical, represent a linear or branched, saturated or unsaturated alkyl group comprising from 6 to 30 carbon atoms, preferably from 8 to 24 carbon atoms and preferentially from 12 to 20 carbon atoms,
A represents a linear or branched alkylene radical group containing from 2 to 8 carbon atoms,
x and y are integers ranging from 1 to 5000; preferably, x ranges from 10 to 2000 and especially from 100 to 1000; preferably, y ranges from 1 to 100.

Preferably, A comprises from 3 to 6 carbon atoms, in particular 4 carbon atoms; preferably, A is branched. A may be a divalent radical chosen from: —CH₂CH₂CH₂— and —CH₂CH(CH₃)CH₂—.

Preferably, R1 and R2, which may be identical or different, represent a saturated linear alkyl group comprising from 6 to 30 carbon atoms, preferentially from 8 to 24 carbon atoms and especially from 12 to 20 carbon atoms, for instance a dodecyl, tetradecyl, pentadecyl, hexadecyl, heptadecyl, octadecyl, nonadecyl or eicosyl group. Advantageously, R1 and R2 represent a mixture of hexadecyl (cetyl) and octadecyl (stearyl) radicals (mixture also known as cetearyl).

Preferentially, for the amodimethicone of formula (K):
x ranges from 10 to 2000 and especially from 100 to 1000;
y ranges from 1 to 100;
A comprises from 3 to 6 carbon atoms, and in particular 4 carbon atoms; preferably, A is branched; preferentially, A is chosen from the divalent radicals: —CH₂CH₂CH₂— and —CH₂CH(CH₃)CH₂—; and
R1 and R2, which may be identical or different, represent a saturated linear radical comprising from 6 to 30 carbon atoms, preferably from 8 to 24 carbon atoms and especially from 12 to 20 carbon atoms, for instance a dodecyl, tetradecyl, pentadecyl, hexadecyl, heptadecyl, octadecyl, nonadecyl or eicosyl group. Advantageously, R1 and R2 represent a mixture of hexadecyl (cetyl) and octadecyl (stearyl) radicals (mixture also known as cetearyl).

As amodimethicone of formula (K), use may be made of bis-cetearyl amodimethicone (INCI name), especially the product sold under the name Silsoft® AX by the company Momentive Performance Materials.

The polyether amines known especially under the reference Jeffamine® from the company Huntsman; and especially:

polyethylene glycol and/or polypropylene glycol a,w-diamines (bearing an amine function at the end of the chain), which may comprise from 2 to 80 units derived from propylene oxide, or which may comprise from 2 to 50 units derived from ethylene oxide and from 1 to 10 units derived from propylene oxide, for instance the products sold under the names Jeffamine® D-230, D-400, D-2000, D-4000, ED-600, ED-9000, ED-2003; polytetrahydrofuran (or polytetramethylene glycol) α,ω-diamines; polybutadiene α,ω-diamines; polyamidoamine (PANAM) dendrimers bearing amine end functions;

poly(meth)acrylates or poly(meth)acrylamides bearing primary or secondary amine side functions, such as poly(3-aminopropyl)methacrylamide or poly(2-aminoethyl) methacrylate.

As amine-based polymer, use is preferably made of polydimethylsiloxanes comprising primary amine groups at the chain end or on side chains.

Preferentially, polydimethylsiloxanes comprising aminopropyl end groups at the chain end are used.

Advantageously, the polyamine compounds used in the process according to the invention are chosen from ethylenediamine, polydimethylsiloxanes comprising primary amine groups at the chain end or on side chains, amodimethicones of formula (K), in particular bis-cetearyl amodimethicone; polyethylene glycol and/or polypropylene glycol α,ω-diamines; ethylenediamine, 1,3-propylenediamine, 1,4-butylenediamine, preferably ethylenediamine.

Preferentially, the polyamine compounds used in the process according to the invention are chosen from ethylenediamine, polydimethylsiloxanes comprising aminopropyl end groups at the chain end, bis-cetearyl amodimethicone, polyethylene glycol/polypropylene glycol α,ω-diamine copolymers comprising from 2 to 50 units derived from ethylene oxide and from 1 to 10 units derived from propylene oxide.

Advantageously, the polyamine compound is used in a mole ratio of amine group of the polyamine compound/maleic anhydride group of the acrylic polymer ranging from 0.01 to 10, preferably ranging from 0.1 to 5, preferentially ranging from 0.1 to 2 and more preferentially ranging from 0.1 to 1.

On contact with the acrylic polymer, the polyamine compound reacts with the maleic anhydride functions to form a crosslinked polymer, for example in the following manner:

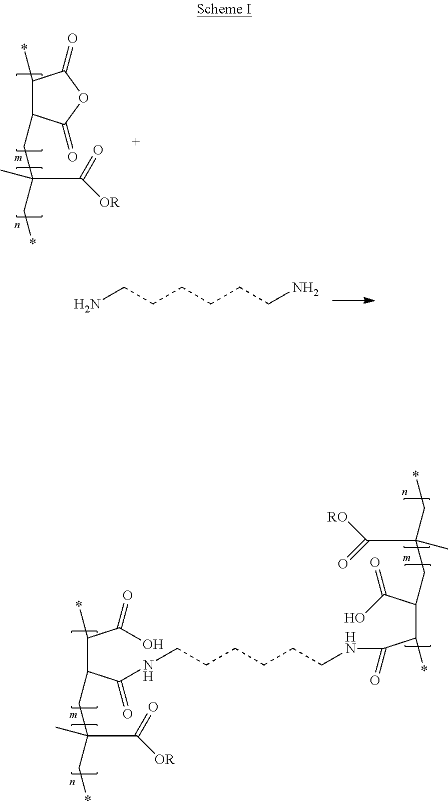

Such a crosslinked polymer is novel and thus also forms the subject of the present invention.

The crosslinked polymer may thus be obtained by reacting said amine compound with the maleic anhydride acrylic polymer described previously. Some or all of the anhydride groups react with the NH or NH₂ group of the polyamine compound and form a unit bearing an amide group and a carboxylic acid group as described in scheme I.

The composition(s) used according to the invention are generally suitable for topical application to keratin materials, and thus generally comprise a physiologically acceptable medium, i.e. a medium that is compatible with the skin and/or its integuments. It is preferably a cosmetically acceptable medium, i.e. a medium which has a pleasant colour, odour and feel and which does not cause any unacceptable discomfort (stinging, tautness or redness) liable to discourage the consumer from using this composition.

Advantageously, the process according to the invention is performed under ambient conditions, in particular at an ambient temperature that may range from 15° C. to 30° C., preferably ranging from 18° C. to 25° C.

According to a preferred embodiment of the invention, the composition comprising the maleic anhydride acrylic polymer may contain a hydrocarbon-based oil.

The hydrocarbon-based oil is an oil that is liquid at room temperature (25° C.).

The term “hydrocarbon-based oil” means an oil formed essentially from, or even constituted of, carbon and hydrogen atoms, and optionally oxygen and nitrogen atoms, and not containing any silicon or fluorine atoms. It may contain alcohol, ester, ether, carboxylic acid, amine and/or amide groups.

The hydrocarbon-based oil may be volatile or non-volatile.

The hydrocarbon-based oil may be chosen from:

hydrocarbon-based oils containing from 8 to 14 carbon atoms, and especially:
branched C₈-C₁₄ alkanes, for instance C₈-C₁₄ isoalkanes of petroleum origin (also known as isoparaffins), for instance isododecane (also known as 2,2,4,4,6-pentamethylheptane), isodecane and, for example, the oils sold under the trade name Isopar or Permethyl,
linear alkanes, for instance n-dodecane (C12) and n-tetradecane (C14) sold by Sasol under the respective references Parafol 12-97 and Parafol 14-97, and also mixtures thereof, the undecane-tridecane mixture, the mixtures of n-undecane (C11) and of n-tridecane (C13) obtained in Examples 1 and 2 of patent application WO 2008/155 059 from the company Cognis, and mixtures thereof,
short-chain esters (containing from 3 to 8 carbon atoms in total) such as ethyl acetate, methyl acetate, propyl acetate or n-butyl acetate,
hydrocarbon-based oils of plant origin such as triglycerides consisting of fatty acid esters of glycerol, the fatty acids of which may have chain lengths varying from C₄ to C₂₄, these chains possibly being linear or branched, and saturated or unsaturated; these oils are especially heptanoic or octanoic acid triglycerides, or alternatively wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion-flower oil and musk rose oil; shea butter; or else caprylic/capric acid triglycerides, for instance those sold by the company Stearineries Dubois or those sold under the names Miglyol 810®, 812® and 818® by the company Dynamit Nobel,
synthetic ethers having from 10 to 40 carbon atoms;
linear or branched hydrocarbons of mineral or synthetic origin, such as petroleum jelly, polydecenes, hydrogenated polyisobutene such as Parleam®, squalane and liquid paraffins, and mixtures thereof,
synthetic esters such as oils of formula R₁COOR₂ in which R₁ represents a linear or branched fatty acid residue containing from 1 to 40 carbon atoms and R₂ represents an, in particular, branched hydrocarbon-based chain containing from 1 to 40 carbon atoms, on the condition that R₁+R₂≥10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, 2-hexyldecyl laurate, 2-octyldecyl palmitate, 2-octyldodecyl myristate, alkyl or polyalkyl heptanoates, octanoates, decanoates or ricinoleates such as propylene glycol dioctanoate; hydroxylated esters such as isostearyl lactate, diisostearyl malate and 2-octyldodecyl lactate; polyol esters and pentaerythritol esters,
fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2-undecylpentadecanol.

Advantageously, the hydrocarbon-based oil is apolar (thus formed solely from carbon and hydrogen atoms).

The hydrocarbon-based oil is preferably chosen from hydrocarbon-based oils containing from 8 to 14 carbon atoms, in particular the apolar oils described previously.

Preferentially, the hydrocarbon-based oil is isododecane.

The composition comprising the polymer may contain, in addition to the hydrocarbon-based oil, a silicone oil. The term “silicone oil” means an oil comprising at least one silicon atom and especially at least one Si—O group. The silicone oil may be volatile or non-volatile.

The term “volatile oil” means an oil (or non-aqueous medium) that is capable of evaporating on contact with the skin in less than one hour, at room temperature and at atmospheric pressure. The volatile oil is a volatile cosmetic oil, which is liquid at room temperature, especially having a non-zero vapour pressure, at room temperature and atmospheric pressure, in particular having a vapour pressure ranging from 0.13 Pa to 40 000 Pa (10⁻³ to 300 mmHg), preferably ranging from 1.3 Pa to 13 000 Pa (0.01 to 100 mmHg) and preferentially ranging from 1.3 Pa to 1,300 Pa (0.01 to 10 mmHg). The term “non-volatile oil” means an oil with a vapour pressure of less than 0.13 Pa.

Volatile silicone oils that may be mentioned include volatile linear or cyclic silicone oils, especially those with a viscosity ≤8 centistokes (cSt) (8×10⁻⁶ m²/s), and especially having from 2 to 10 silicon atoms and in particular from 2 to 7 silicon atoms, these silicones optionally comprising alkyl or alkoxy groups having from 1 to 10 carbon atoms. As volatile silicone oil that may be used in the invention, mention may be made especially of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

As non-volatile silicone oils, mention may be made of linear or cyclic non-volatile polydimethylsiloxanes (PDMSs); polydimethylsiloxanes comprising alkyl, alkoxy or phenyl groups, which are pendant or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates.

Advantageously, the composition may comprise a hydrocarbon-based oil in a content ranging from 60% to 100% by weight relative to the total weight of the oils present in the composition and from 0 to 40% by weight of silicone oil. According to a preferred embodiment of the invention, the composition contains as oil only a hydrocarbon-based oil.

The composition according to the invention may comprise a cosmetic additive chosen from fragrances, preserving agents, fillers, UV-screening agents, oils, waxes, surfactants, moisturizers, vitamins, ceramides, antioxidants, free-radical scavengers, polymers, thickeners and dyestuffs.

The composition according to the invention may also comprise a dyestuff such as pulverulent dyestuffs, liposoluble dyes or water-soluble dyes. This dyestuff may be present in a content ranging from 0.01% to 30% by weight, relative to the total weight of the composition.

The pulverulent dyestuffs may be chosen from pigments and nacres.

The pigments may be white or coloured, mineral and/or organic, and coated or uncoated. Among the mineral pigments that may be mentioned are titanium dioxide, optionally surface-treated, zirconium, zinc or cerium oxide, and also iron or chromium oxide, manganese violet, ultramarine blue, chromium hydrate and ferric blue. Among the organic pigments that may be mentioned are carbon black, pigments of D&C type and lakes based on cochineal carmine or on barium, strontium, calcium or aluminium.

The nacres may be chosen from white nacreous pigments such as mica coated with titanium or with bismuth oxychloride, coloured nacreous pigments such as titanium mica with iron oxides, titanium mica with in particular ferric blue or chromium oxide, titanium mica with an organic pigment of the abovementioned type, and also nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto. The water-soluble dyes are, for example, beetroot juice or methylene blue.

Advantageously, the composition according to the invention is a skincare composition.

The composition according to the invention may be a makeup composition such as a foundation, a lipstick or a liner.

According to one embodiment, the composition according to the invention is a makeup composition and comprises a volatile oil and a non-volatile oil as described previously. In particular, the makeup composition may comprise a hydrocarbon-based volatile oil and a hydrocarbon-based non-volatile oil.

According to one embodiment, the composition according to the invention is a hair fixing composition.

According to one embodiment, the composition used in the process according to the invention is an anhydrous composition. The term “anhydrous composition” means a composition containing less than 2% by weight of water, or even less than 0.5% of water, and is especially free of water. Where appropriate, such small amounts of water may especially be introduced by ingredients of the composition that may contain residual amounts thereof.

According to a first embodiment of the process according to the invention, a composition, especially a cosmetic composition, comprising the acrylic polymer is first applied to the keratin materials, and said polyamine compound or a cosmetic composition containing same is then applied. The application of the polyamine compound may be performed after a time of between 5 minutes and one hour after having applied the acrylic polymer to the keratin materials.

According to a second embodiment of the process according to the invention, said polyamine compound, or a cosmetic composition containing same, is first applied to the keratin materials, and the composition, especially the cosmetic composition, comprising the acrylic polymer is then applied. The application of the acrylic polymer may be performed after a time of between 5 minutes and one hour after having applied said polyamine compound to the keratin materials.

The invention will now be described with reference to the examples that follow, which are given as non-limiting illustrations.

### EXAMPLE 1

### Isobornyl Acrylate/2-Ethylhexyl Acrylate/Maleic Anhydride Copolymer (70/20/10 by Weight)—Polymer 1

70 g of isobornyl acrylate, 20 g of 2-ethylhexyl acrylate and 10 g of maleic anhydride were placed in a jacketed 1-litre reactor equipped with a stirring anchor. A mixture of 70 g of isododecane and 30 g of ethyl acetate was then added. The medium was brought to a temperature of 40° C. with stirring (150 rpm) and was sparged with argon for 10 minutes, followed by addition of 0.5 g of initiator tert-butyl peroxy-2-ethylhexanoateTrigonox 21S (Trigonox® 21S from Akzo Nobel).

The heating of the jacket was set at 90° C. for 7 hours at 150 rpm.

The medium was then diluted with 300 g of isododecane, and then concentrated by distillation to remove the ethyl acetate and the unreacted maleic anhydride.

A solution containing 30% by weight of the copolymer in isododecane (yield of greater than 90%) was finally obtained.

The polymer obtained has a molecular weight (Mw) of close to 200 000 g/mol.

### EXAMPLE 2

### Isobornyl Acrylate/2-Ethylhexyl Acrylate/Maleic Anhydride Copolymer (65/25/10 by Weight)—Polymer 2

The polymer was prepared according to the procedure of Example 1, using 65 g of isobornyl acrylate, 25 g of 2-ethylhexyl acrylate and 10 g of maleic anhydride.

A solution containing 30% by weight of the copolymer in isododecane (yield of greater than 90%) was finally obtained.

The polymer obtained has a molecular weight (Mw) of close to 200 000 g/mol.

### EXAMPLE 3

### Isobornyl Acrylate/2-Ethylhexyl Acrylate/Maleic Anhydride Copolymer (75/20/5 by Weight)—Polymer 3

The polymer was prepared according to the procedure of Example 1, using 75 g of isobornyl acrylate, 20 g of 2-ethylhexyl acrylate and 5 g of maleic anhydride.

A solution containing 30% by weight of the copolymer in isododecane (yield of greater than 90%) was finally obtained.

The polymer obtained has a molecular weight (Mw) of close to 200 000 g/mol.

### EXAMPLE 4

### Isobornyl Acrylate/2-Ethylhexyl Acrylate/Maleic Anhydride Copolymer (60/20/20 by Weight)—Polymer 4

The polymer was prepared according to the procedure of Example 1, using 60 g of isobornyl acrylate, 20 g of 2-ethylhexyl acrylate and 20 g of maleic anhydride. A solution containing 36% by weight of the copolymer in isododecane (yield of greater than 90%) was finally obtained.

The polymer obtained has a molecular weight (Mw) of close to 200 000 g/mol.

### COMPARATIVE EXAMPLES 5 TO 10

### Cosmetic Evaluation of Makeup Compositions

6 makeup compositions (lipstick, foundation) of base coat containing the polymer of Example 1 and of top coat described below (Examples 6, 8, 10 according to the invention: top coat with 3-aminopropyl-terminated polydimethylsiloxane; Examples 5, 7, 9, 13 outside the invention: top coat without 3-aminopropyl-terminated polydimethylsiloxane) were prepared.

Each base coat composition was applied onto a skin equivalent support made of elastomer by producing a deposit with a wet thickness of 100 μm, which was left to dry at room temperature (25° C.) for 24 hours.

The top coat composition was then applied onto each dry base coat deposit by producing a deposit with a wet thickness of 100 μm, which was left to dry at room temperature (25° C.) for 24 hours.

The state of the film obtained was then observed.

The resistance of the film obtained was evaluated by separately applying 0.5 ml of water, 0.5 ml of olive oil and 0.5 ml of sebum; after 5 minutes of contact, the surface of the film was rubbed with cotton wool and the state of the film was then observed.

The tackiness of the film and its capacity for transferring or not transferring on touching the film with a finger were also evaluated.

The evaluation was made in the following manner:

+++: very efficient evaluated cosmetic property

++: moderately efficient evaluated cosmetic property

+: sparingly efficient evaluated cosmetic property

0: inefficient evaluated cosmetic property

The following results were obtained:

| | | Example | | Example | | Example |
|---|---|---|---|---|---|---|
| | Example 5 | 6 (invention) | Example 7 | 8 (invention) | Example 9 | 10 (invention) |
| Base Coat | | | | | | |
| Polymer of | 25 g | 25 g | 20 g | 20 g | 25 g | 25 g |
| Example 1 | | | | | | |
| Pigmentary | 5 g with | 5 g with | 5 g with | 5 g with | 5 g with | 5 g with |
| paste | DC Red 7 | DC Red 7 | DC Red 7 | DC Red 7 | red iron oxide | red iron oxide |
| containing | | | | | | |
| 40% by | | | | | | |
| weight of | | | | | | |
| pigment in | | | | | | |
| isododecane | | | | | | |
| Disteardimonium | 10 g | 10 g | 10 g | 10 g | 10 g | 10 g |
| hectorite | | | | | | |
| (Bentone Gel | | | | | | |
| ISD V from | | | | | | |
| Elementis) | | | | | | |
| 2-Octyldodecanol | | | 20 g | 20 g | | |
| Isododecane | qs 100 g | qs 100 g | qs 100 g | qs 100 g | qs 100 g | qs 100 g |
| Top Coat | No | Yes | No | Yes | No | Yes |
| 3-Aminopropyl- | | 10 g | | 10 g | | 10 g |
| terminated | | | | | | |
| polydimethylsiloxane | | | | | | |
| (Mn 2 500; | | | | | | |
| reference | | | | | | |
| 481688 from | | | | | | |
| Sigma) | | | | | | |
| Isododecane | | 90 g | | 90 g | | 90 g |
| Appearance | Homogeneous | Homogeneous | Homogeneous | Homogeneous | Homogeneous | Homogeneous |
| of the film | film | film | film | film | film | film |
| Water | ++ | +++ | ++ | +++ | ++ | +++ |
| resistance | | | | | | |
| Olive oil | 0 | +++ | 0 | +++ | 0 | +++ |
| resistance | | | | | | |
| Sebum | 0 | +++ | 0 | +++ | 0 | +++ |
| resistance | | | | | | |
| Non-tacky | +++ | +++ | + | +++ | +++ | +++ |
| Transfer- | +++ | +++ | + | +++ | +++ | +++ |
| resistant | | | | | | |

The results obtained show that the deposits resulting from the application of polymer 1 followed by 3-aminopropyl-terminated polydimethylsiloxane (Examples 6, 10) with 2-octyldodecanol (Example 8) form a non-tacky homogeneous film that does not transfer by finger, and that shows better resistance to water, to oil and to sebum, whereas the sole application of polymer 1 (Examples 5, 7, 9) form a deposit that has poor resistance to oil and to sebum. In addition, Example 7 forms a film that is more tacky and that transfers onto the finger.

Thus, the resistance of the film to contact with olive oil and sebum is markedly improved by the application of the top coat composition comprising the 3-aminopropyl-terminated polydimethylsiloxane. Furthermore, when the base coat composition contains 2-octyldodecanol, application of the top coat also makes it possible to improve the tack-free and transfer-resistance properties on contact of the film obtained with a finger.

The lipstick compositions of Examples 6 and 8 applied to the lips thus make it possible to obtain a non-tacky, transfer-resistant and oil- and sebum-resistant makeup which thus has good persistence.

The compositions of Example 10 applied to the skin thus make it possible to obtain a non-tacky, transfer-resistant and oil- and sebum-resistant makeup which thus has good persistence.

### COMPARATIVE EXAMPLES 11 AND 12

### Cosmetic Evaluation of Makeup Compositions

The two base coat makeup compositions (lipstick) containing the polymer of Example 2 and a top coat composition containing 3-aminopropyl-terminated polydimethylsiloxane described below were prepared.

The compositions were applied and the cosmetic properties of the film obtained were evaluated as described previously in Examples 5 to 10.

| | | | Example 12 | |
|---|---|---|---|---|
| | Example 11 | | (invention) | |
| Base Coat | | | | |
| Polymer of Example 2 | 25 | g | 25 | g |
| Pigmentary paste | 5 g with DC Red 7 | | 5 g with DC Red 7 | |
| containing 40% by | | | | |
| weight of pigment in | | | | |
| isododecane | | | | |
| Disteardimonium | 10 | g | 10 | g |
| hectorite | | | | |
| (Bentone Gel ISD V | | | | |
| from Elementis) | | | | |
| Isododecane | qs 100 | g | qs 100 | g |

| | | |
|---|---|---|
| Top Coat | No | Yes |

| | | |
|---|---|---|
| 3-Aminopropyl- | 10 | g |
| terminated | | |
| polydimethylsiloxane | | |
| (Mn 2 500; reference | | |
| 481688 from Sigma) | | |
| Isododecane | 90 | g |

| | | |
|---|---|---|
| Appearance of the film | Homogeneous | Homogeneous |
| | film | film |
| Water resistance | ++ | +++ |
| Olive oil resistance | 0 | +++ |
| Sebum resistance | 0 | +++ |
| Non-tacky | +++ | +++ |
| Transfer-resistant | +++ | +++ |

The results obtained show that the deposit resulting from the application of polymer 2 followed by 3-aminopropyl-terminated polydimethylsiloxane (Example 12) forms a non-tacky homogeneous film that does not transfer by finger, and that is resistant to water, to oil and to sebum, whereas the sole application of polymer 2 (Example 11) forms a deposit that has poor resistance to oil and to sebum.

Thus, the resistance of the film to contact with olive oil and sebum is markedly improved by the application of the top coat composition comprising the 3-aminopropyl-terminated polydimethylsiloxane.

The lipstick compositions of Example 12 applied to the lips thus make it possible to obtain a non-tacky, transfer-resistant and water-, oil- and sebum-resistant makeup which thus has good persistence.

### COMPARATIVE EXAMPLES 13 AND 14

### Cosmetic Evaluation of Makeup Compositions

The two makeup compositions (foundation) of base coat containing the polymer of Example 3 and a top coat composition containing 3-aminopropyl-terminated polydimethylsiloxane described below were prepared.

The compositions were applied and the cosmetic properties of the film obtained were evaluated as described previously in Examples 5 to 10.

| | | | Example 14 | |
|---|---|---|---|---|
| | Example 13 | | (invention) | |
| Base Coat | | | | |
| Polymer of Example 3 | 25 | g | 25 | g |
| Pigmentary paste | 5 g with red iron | | 5 g with red iron | |
| containing 40% by | oxide | | oxide | |
| weight of pigment in | | | | |
| isododecane | | | | |
| Disteardimonium | 10 | g | 10 | g |
| hectorite | | | | |
| (Bentone Gel ISD V | | | | |
| from Elementis) | | | | |
| Isododecane | qs 100 | g | qs 100 | g |

| | | |
|---|---|---|
| Top Coat | No | Yes |

| | | |
|---|---|---|
| 3-Aminopropyl- | 10 | g |
| terminated | | |
| polydimethylsiloxane | | |
| (Mn 2 500; reference | | |
| 481688 from Sigma) | | |
| Isododecane | 90 | g |

| | | |
|---|---|---|
| Appearance of the film | Homogeneous | Homogeneous |
| | film | film |
| Water resistance | ++ | +++ |
| Olive oil resistance | 0 | +++ |
| Sebum resistance | 0 | +++ |
| Non-tacky | +++ | +++ |
| Transfer-resistant | +++ | +++ |

The results obtained show that the deposit resulting from the application of polymer 3 followed by 3-aminopropyl-terminated polydimethylsiloxane (Example 12) forms a non-tacky homogeneous film that does not transfer by finger, and that is resistant to water, to oil and to sebum, whereas the sole application of polymer 2 (Example 11) forms a deposit that has poor resistance to oil and to sebum.

Thus, the resistance of the film to contact with olive oil and sebum is markedly improved by the application of the top coat composition comprising the 3-aminopropyl-terminated polydimethylsiloxane.

The compositions of Example 14 applied to the skin thus make it possible to obtain a non-tacky, transfer-resistant and water-, oil- and sebum-resistant makeup which thus has good persistence.

### COMPARATIVE EXAMPLES 15 AND 16

### Cosmetic Evaluation of Mascara Composition

A base coat composition containing 25% AM of polymer of Example 4, 5% of black iron oxide, 10% of disteardimonium hectorite (Bentone Gel ISD V from Elementis) and 65% of isododecane was applied to a false eyelash specimen. The treated eyelashes were left to dry naturally (25° C.) for 24 hours.

0.5 g of a top coat composition containing 10% AM of 3-aminopropyl-terminated polydimethylsiloxane (Mn 2500; reference 481688 from Sigma) in isododecane was then applied to the false eyelashes, and was then left to dry naturally for 24 hours (Example 16 according to the invention).

The persistence with respect to sebum of the deposit formed on the treated eyelashes was then evaluated by immersing the treated eyelashes in artificial sebum for 5 minutes. The eyelashes were then left to dry naturally and were rubbed on blotting paper. No trace of deposit was observed on the paper: the deposit formed on the eyelashes is thus sebum-resistant.

In comparison, the same test was performed on eyelashes treated with the sole application of the base coat composition (without application of the top coat composition) (Example 15): black traces were observed on the blotting paper: the deposit formed on the eyelashes shows poor sebum resistance.

Application of the top coat composition containing the 3-aminopropyl-terminated polydimethylsiloxane makes it possible to improve the sebum resistance of the film obtained.

### COMPARATIVE EXAMPLES 17 AND 18

### Cosmetic Evaluation of Hair Composition

0.5 g of a base coat composition containing 10% AM of the polymer of Example 1 in isododecane was applied to a 2.7 g lock of washed and dried hair (lock No. 1). The treated lock was left to dry naturally (25° C.) for 24 hours. 0.5 g of a top coat composition containing 10% AM of ethylenediamine in isododecane was then applied to the lock, which was then left to dry naturally for 24 hours (Example 18 according to the invention). The base coat composition alone was applied to another lock of hair (lock No. 2) (Example 17).

The fixing quality of the lock of hair was evaluated by observing the more or less rigid appearance of the lock: the lock is taken by one of its ends with the fingers and turned upside-down, holding it at the bottom; the shape of the lock is then observed; either the lock retains its shape, which means that the lock is fixed very well; or the lock becomes deformed (under the effect of gravity) which means that the lock is not fixed well.

The persistence with respect to water and the fixing property of the treated locks were then evaluated by immersing the treated lock in water for 5 minutes. The locks were then dried manually, followed by drying under a hood. The rigidity of the two locks was observed.

It was found that the lock treated according to Example 18 before and after immersion in water has a rigid form with good hair fixing.

The hair fixing of Example 18 thus shows good persistence with respect to water.

The lock treated according to Example 17 has a rigid form before immersion in the water, but loses its rigidity after immersion in the water: the lock is supple and has no fixing.

### COMPARATIVE EXAMPLES 19 TO 24

### Cosmetic Evaluation of Makeup Compositions with Application in Two Steps

The makeup composition (lipstick) of base coat containing the polymer of Example 4 and 5 top coat compositions containing an amine compound chosen from 3-aminopropyl-terminated polydimethylsiloxane (Mn=25 000 and 50 000), ethylenediamine, polyetherdiamine and bis-cetearyl amodimethicone described below were prepared.

The compositions were applied and the cosmetic properties of the film obtained were evaluated as described previously in Examples 5 to 10.

The following results were obtained:

| | | Example | Example | Example | Example | Example |
|---|---|---|---|---|---|---|
| | Example 19 | 20 (invention) | 21 (invention) | 22 (invention) | 23 (invention) | 24 (invention) |
| Base Coat | | | | | | |
| Polymer of | 20 g AM | 20 g AM | 20 g AM | 20 g AM | 20 g AM | 20 g AM |
| Example 4 | | | | | | |
| Pigmentary | 5 g with | 5 g with | 5 g with | 5 g with | 5 g with | 5 g with |
| paste containing | DC Red | DC Red | DC Red | DC Red | DC Red | DC Red |
| 40% by weight of | 7 | 7 | 7 | 7 | 7 | 7 |
| pigment in | | | | | | |
| isododecane | | | | | | |
| Disteardimonium | 10 g | 10 g | 10 g | 10 g | 10 g | 10 g |
| hectorite | | | | | | |
| (Bentone Gel | | | | | | |
| ISD V from | | | | | | |
| Elementis) | | | | | | |
| Isododecane | qs 100 g | qs 100 g | qs 100 g | qs 100 g | qs 100 g | qs 100 g |
| Top Coat | | | | | | |
| 3-Aminopropyl- | | 10 g | | | | |
| terminated | | | | | | |
| polydimethylsiloxane | | | | | | |
| (Mn 25 000; DMS | | | | | | |
| A-31 from Gelest) | | | | | | |
| 3-Aminopropyl- | | | 10 g | | | |
| terminated | | | | | | |
| polydimethylsiloxane | | | | | | |
| (Mn 25 000; DMS | | | | | | |
| A-35 from Gelest) | | | | | | |
| Ethylenediamine | | | | 10 g | | |
| Polyetherdiamine | | | | | 10 g | |
| (1) | | | | | | |
| Bis-cetearyl | | | | | | 10 g |
| amodimethicone | | | | | | |
| (2) | | | | | | |
| Isododecane | | qs 100 g | qs 100 g | qs 100 g | qs 100 g | qs 100 g |
| Evaluation of | | | | | | |
| the film | | | | | | |
| Appearance of | Homogeneous | Homogeneous | Homogeneous | Homogeneous | Homogeneous | Homogeneous |
| the film | film | film | film | film | film | film |
| Olive oil | 0 | +++ | ++ | ++ | ++ | ++ |
| resistance | | | | | | |
| Non-tacky | +++ | +++ | +++ | +++ | +++ | +++ |
| Transfer- | ++ | +++ | +++ | +++ | +++ | +++ |
| resistant | | | | | | |

(1) Jeffamine ® ED-900 Polyetheramine (Huntsman)
(2) Silsoft ® AX (Momentive Performance Materials)

The results obtained show that the deposit resulting from the application of polymer 4 followed by the amine compound (Examples 20 to 24) forms a non-tacky homogeneous film that does not transfer by finger, and that is resistant to oil, whereas the sole application of polymer 4 (Example 19) forms a deposit that transfers onto the finger and has poor resistance to oil.

Thus, the non-tacky and transfer-resistant aspect on contact with the finger, and also the resistance of the film to contact with olive oil are improved with the application of the top coat composition containing the amine compounds tested.

The compositions of Examples 20 to 24 applied to the lips thus make it possible to obtain a non-tacky, transfer-resistant and oil-resistant makeup which thus has good persistence.

1. A cosmetic process for treating keratin materials, comprising the sequential application to the keratin materials of a cosmetic composition comprising a maleic anhydride acrylic polymer and a polyamine compound or a cosmetic composition containing same,
said maleic anhydride acrylic polymer being obtained by polymerization of:
(a) 50% to 90% by weight, relative to the total weight of monomers, of isobornyl (meth)acrylate
(b) 1% to 50% by weight of maleic anhydride
(c) 0 to 49% by weight of additional (meth)acrylate monomer chosen from:
(i) linear or branched, saturated or unsaturated C₁-C₂₀ alkyl (meth)acrylates, optionally interrupted with one or more non-adjacent heteroatoms chosen from O and S or with a group NR, R being a C₁-C₄ alkyl group, optionally substituted with a phenyl or furfuryl group;
(ii) saturated C₄-C₈ cycloalkyl (meth)acrylates optionally interrupted with O or NH;
said polyamine compound bearing several primary amine and/or secondary amine groups, the polyamine compound not being an alkoxysilane.

2. The process according to claim 1, wherein said maleic anhydride acrylic polymer is derived from the polymerization of:
(a) 50% to 80% by weight, relative to the total weight of monomers, of isobornyl (meth)acrylate
(b) 5% to 30% by weight of maleic anhydride
(c) 15% to 30% by weight of said additional (meth)acrylate monomer.

3. The process according to claim 1, wherein said maleic anhydride acrylic polymer is derived from the polymerization of:
(a) 50% to 80% by weight, relative to the total weight of monomers, of isobornyl (meth)acrylate
(b) 5% to 25% by weight of maleic anhydride
(c) 15% to 30% by weight of said additional (meth)acrylate monomer.

4. The process according to claim 1, wherein the maleic anhydride acrylic polymer is derived from the polymerization of:
(a) 50% to 80% by weight, relative to the total weight of monomers, of isobornyl (meth)acrylate
(b) 5% to 15% by weight of maleic anhydride
(c) 15% to 30% by weight of said additional (meth)acrylate monomer.

5. The process according to claim 1, wherein the maleic anhydride acrylic polymer is derived from the polymerization of:
(a) 60% to 80% by weight, relative to the total weight of monomers, of isobornyl (meth)acrylate
(b) 5% to 12% by weight of maleic anhydride
(c) 15% to 30% by weight of said additional (meth)acrylate monomer.

6. The process according to claim 1, wherein said additional (meth)acrylate monomer is chosen from C₆-C₁₆ alkyl (meth)acrylates.

7. The process according to claim 1, wherein said maleic anhydride acrylic polymer is derived from the polymerization of:
(a) 50% to 80% by weight, relative to the total weight of monomers, of isobornyl (meth)acrylate
(b) 5% to 30% by weight of maleic anhydride
(c) 15% to 30% by weight of C₆-C₁₆ alkyl acrylate monomer.

8. The process according to claim 1, wherein the maleic anhydride acrylic polymer is derived from the polymerization of:
(a) 50% to 80% by weight, relative to the total weight of monomers, of isobornyl (meth)acrylate
(b) 5% to 25% by weight of maleic anhydride
(c) 15% to 30% by weight of C₆-C₁₆ alkyl acrylate monomer
and in that it is preferably derived from the polymerization of:
(a) 50% to 80% by weight, relative to the total weight of monomers, of isobornyl (meth)acrylate
(b) 5% to 15% by weight of maleic anhydride
(c) 15% to 30% by weight of C₆-C₁₆ alkyl acrylate monomer.

9. The process according to claim 1, wherein the acrylic polymer comprises isobornyl acrylate, 2-ethylhexyl acrylate and maleic anhydride.

10. The process according to claim 1 wherein the acrylic polymer has a weight-average molecular weight ranging from 5000 to 1 000 000 g/mol.

11. The process according to claim 1 wherein the polyamine compound comprises from 2 to 20 carbon atoms.

12. The process according to claim 1, wherein the polyamine compound is chosen from N-methyl-1,3-diaminopropane, N-propyl-1,3-diaminopropane, N-isopropyl-1,3-diaminopropane, N-cyclohexyl-1,3-diaminopropane, 2-(3-aminopropylamino)ethanol, 3-(2-aminoethyl)aminopropylamine, bis(3-aminopropyl)amine, methylbis(3-aminopropyl)amine, N-(3-aminopropyl)-1,4-diaminobutane, N,N-dimethyldipropylenetriamine, 1,2-bis(3-aminopropylamino)ethane, N,N′-bis(3-aminopropyl)-1,3-propanediamine, ethylenediamine, 1,3-propylenedimaine, 1,4-butylenediamine, lysine, cystamine, xylenediamine, tris(2-aminoethyl)amine and spermidine.

13. The process according to claim 1, wherein the polyamine compound is chosen from amine-based polymers, especially having a weight-average molecular weight ranging from 500 to 1 000 000.

14. The process according to claim 13, wherein the polyamine compound is chosen from poly((C₂-C₅)alkyleneimines); polyamino acids bearing NH₂ groups; amino polyvinyl alcohol, acrylamidopropylamine-based copolymers; chitosans;
polydimethylsiloxanes comprising primary amine groups at the chain end or on side chains; 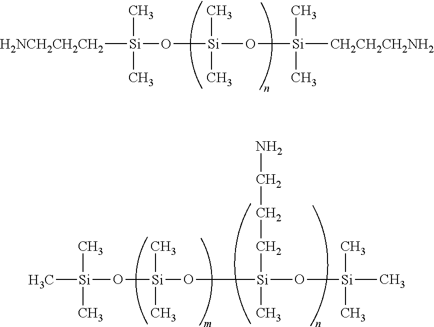
amodimethicones of formula (D): 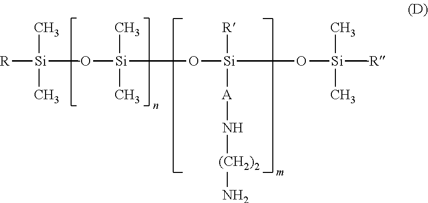
in which R, R′ and R″, which may be identical or different, each represent a C₁-C₄ alkyl or hydroxyl group, A represents a C₃ alkylene group and m and n are such that the weight-average molecular mass of the compound is between 5000 and 500 000 approximately;
amodimethicones of formula (K): 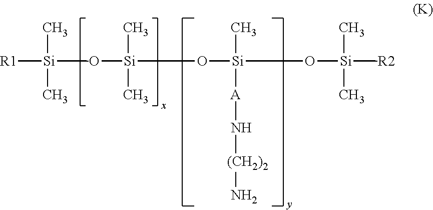
in which:
R1 and R2, which may be identical or different represent a linear or branched, saturated or unsaturated alkyl group comprising from 6 to 30 carbon atoms,
A represents a linear or branched alkylene radical group containing from 2 to 8 carbon atoms,
x and y are integers ranging from 1 to 5000;
polyetherdiamines;
polyamidoamine dendrimers bearing amine end functions;
poly(meth)acrylates or poly(meth)acrylamides bearing primary or secondary amine side functions.

15. The process according to claim 1, wherein the polyamine compound is used in a mole ratio of the amine group of the polyamine compound/maleic anhydride group of the acrylic polymer ranging from 0.01 to 10.

16. The process according to claim 1, wherein the acrylic polymer is present in a content ranging from 0.1% to 40% by weight, relative to the total weight of the composition.

17. The process according to claim 1, wherein the composition comprises a hydrocarbon-based oil.

18. The process according to claim 1, wherein the composition comprising the maleic anhydride acrylic polymer is first applied to the keratin materials, and the polyamine compound or a composition containing same and comprising a physiologically acceptable medium is then applied.

19. The process according to claim 1, wherein the amine compound, or a composition containing same and comprising a physiologically acceptable medium, is first applied to the keratin materials, and the composition comprising the maleic anhydride acrylic polymer is then applied.

20. The process according to claim 1, wherein it is performed on the skin, the lips, the eyelashes, the hair or the nails.

21. A kit comprising a first composition comprising a maleic anhydride acrylic polymer obtained by polymerization of:
(a) 50% to 90% by weight, relative to the total weight of monomers, of isobornyl (meth)acrylate
(b) 1% to 50% by weight of maleic anhydride
(c) 0 to 49% by weight of additional (meth)acrylate monomer chosen from:
(i) linear or branched, saturated or unsaturated C₁-C₂₀ alkyl (meth)acrylates, optionally interrupted with one or more non-adjacent heteroatoms chosen from O and S or with a group NR, R being a C₁-C₄ alkyl group, optionally substituted with a phenyl or furfuryl group;
(ii) saturated C₄-C₈ cycloalkyl (meth)acrylates optionally interrupted with O or NH and comprising a physiologically acceptable medium, and a second composition comprising a polyamine compound bearing several primary amine and/or secondary amine groups, the polyamine compound not being an alkoxysilane and comprising a physiologically acceptable medium, the first and second compositions each being packaged in a separate packaging assembly.

22. A polymer obtained by reacting a maleic anhydride acrylic polymer obtained by polymerization of:
(a) 50% to 90% by weight, relative to the total weight of monomers, of isobornyl (meth)acrylate
(b) 1% to 50% by weight of maleic anhydride
(c) 0 to 49% by weight of additional (meth)acrylate monomer chosen from:
(i) linear or branched, saturated or unsaturated C₁-C₂₀ alkyl (meth)acrylates, optionally interrupted with one or more non-adjacent heteroatoms chosen from O and S or with a group NR, R being a C₁-C₄ alkyl group, optionally substituted with a phenyl or furfuryl group;
(ii) saturated C₄-C₈ cycloalkyl (meth)acrylates optionally interrupted with O or NH with a polyamine compound bearing several primary amine and/or secondary amine groups, the polyamine compound not being an alkoxysilane.
